(19) United States
(12) Patent Application Publication
(10) Pub. No.: US 2022/0040358 A1
(43) Pub. Date: Feb. 10, 2022

(54) Ultra-v mask

(71) Applicant: Paul Oddo, Palm Bay, FL (US)

(72) Inventor: Paul Oddo, Palm Bay, FL (US)

(21) Appl. No.: 16/873,879

(22) Filed: Aug. 4, 2020

(51) Int. Cl. A61L 9/20; A62B 18/02; A62B 18/08; A62B 18/10

(52) U.S. Cl. CPC A61L 9/20; A62B 18/025; A61L 2209/12; A62B 18/10; A62B 18/08

(57) ABSTRACT

As the human population continues to grow and evolve so are the viruses and bacterium. Our modern world is becoming a more complex and hazardous environment as time moves on. Through technology we must create counter measures to combat this growing threat. One such measure is the creation of the Ultra-V Mask. This mask is state of the art and like none other. My personal protection device treats the air you inhale and exhale with uniquely designed baffled chambers that utilize UVC light as a sterilization method limiting if not eliminating the risk of transmission of harmful microbes to the user or others. As a result The Ultra-V Mask may very well save your life and lives of others.

### BRIEF SUMMARY

### Background of the Invention

Since the dawn of mankind masks of all shapes and sizes have been created over the years they have been designed to perform many functions. Masks were first originated during ritualistic ceremonies and in tribal warfare during humans prehistoric days. As time went on masks became a little more complex and used for a wider variety of applications. We now have chemical masks, clinical masks, dust masks and more. A mask is a cover or partial covering of the face designed to protect the user from projectiles or harmful contaminants in our environment. They are worn in a variety of ways with different fastening methods covering the mouth and nose. All masks perform the same basic function and that is the filtration of air the human lung processes.

### SUMMARY OF THE INVENTION

The present invention relates to saving lives by providing the general population as well as others with a device that protects the user and bystander from harmful airborne contaminants and microbes. There are a variety of masks available on the modern market today but all do not provide complete protection for the user and non user from harmful bacteria. The Ultra-V Mask provides this protection as outlined in the body of this document. The Ultra-V Mask is virtually a mobile air treatment product that is worn on the face that utilizes simple UVC light to destroy airborne bacteria upon inhalation and exhalation. There have been notable products developed over the years to treat air we breath in buildings with light but no products were developed to treat the air we breath while on the move. The Ultra-V mask has finally arrived as the solution to this problem. The objective of the present invention is to provide personal protection for the people of the world by treating and filtering the air that is inhaled and exhaled by the human lung with UVC light.

### DESCRIPTION OF DRAWINGS

FIG. 1 Depicts an assembled front view and side view of the Ultra V Mask.

FIG. 2 Illustrates a side view of the light chamber depicting the internal workings and dimensions of the light chamber.

FIG. 3 Illustrates the front view of both light chambers and air flow direction along with dimensions.

FIG. 4 Depicts the premanufactured face shroud coupled to the harness and method of attachment to the face.

FIG. 5 Depicts the premanufactured check valve used to establish airflow.

### DETAILED DESCRIPTION: DESCRIPTION OF THE PREFERRED EMBODIMENTS

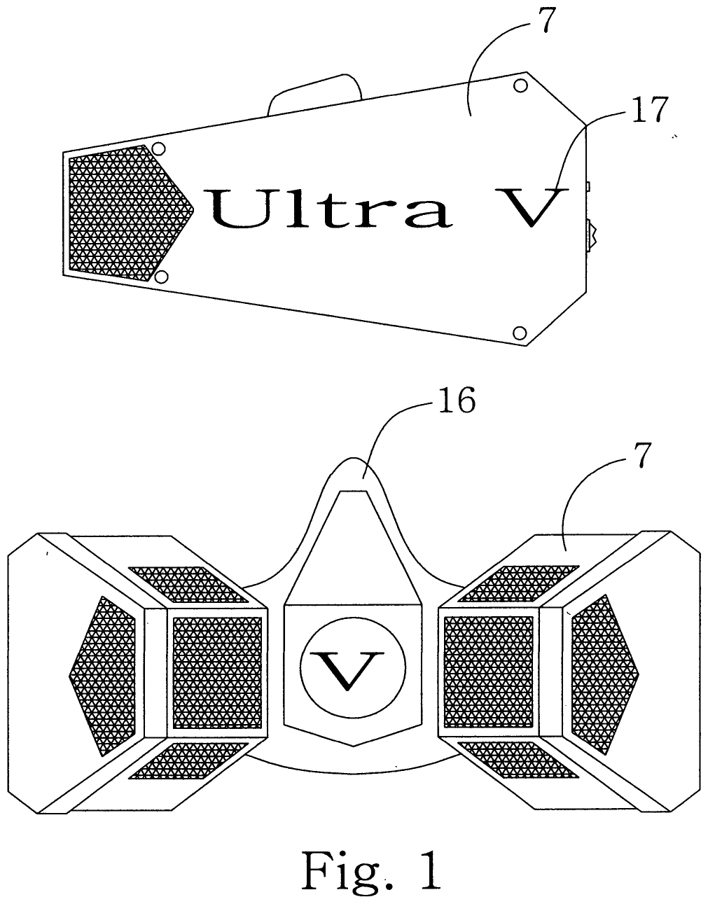

FIG. 1 provides the visual front and side view of the assembled mask with both light canisters (7) attached to the face shroud (16) and the product insignia placement upon the light chambers. The insignia (Ultra V)(17) illuminates when unit is activated indicating system is functional.

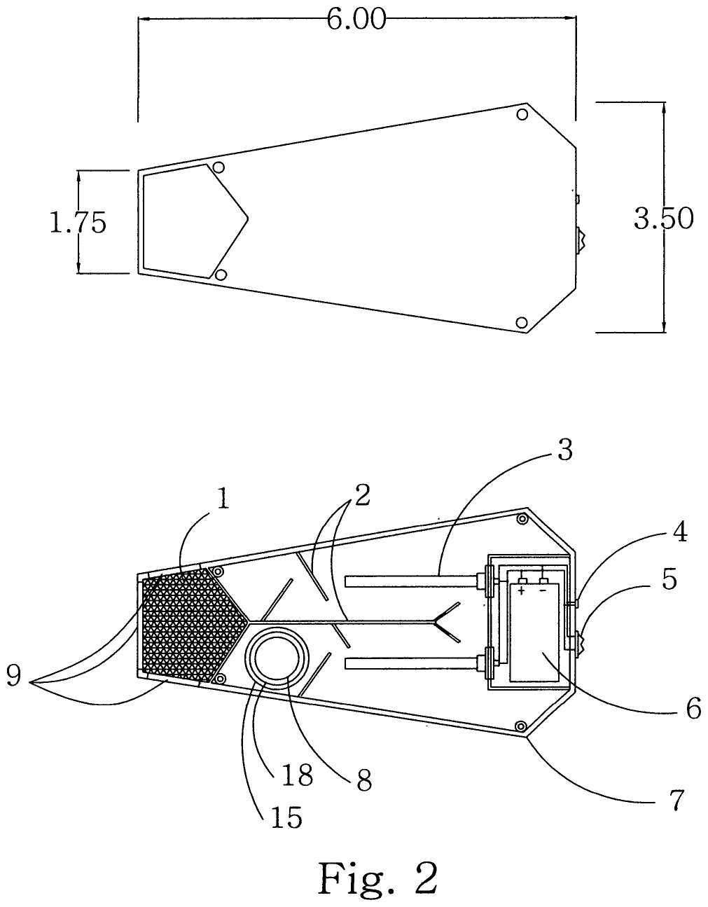

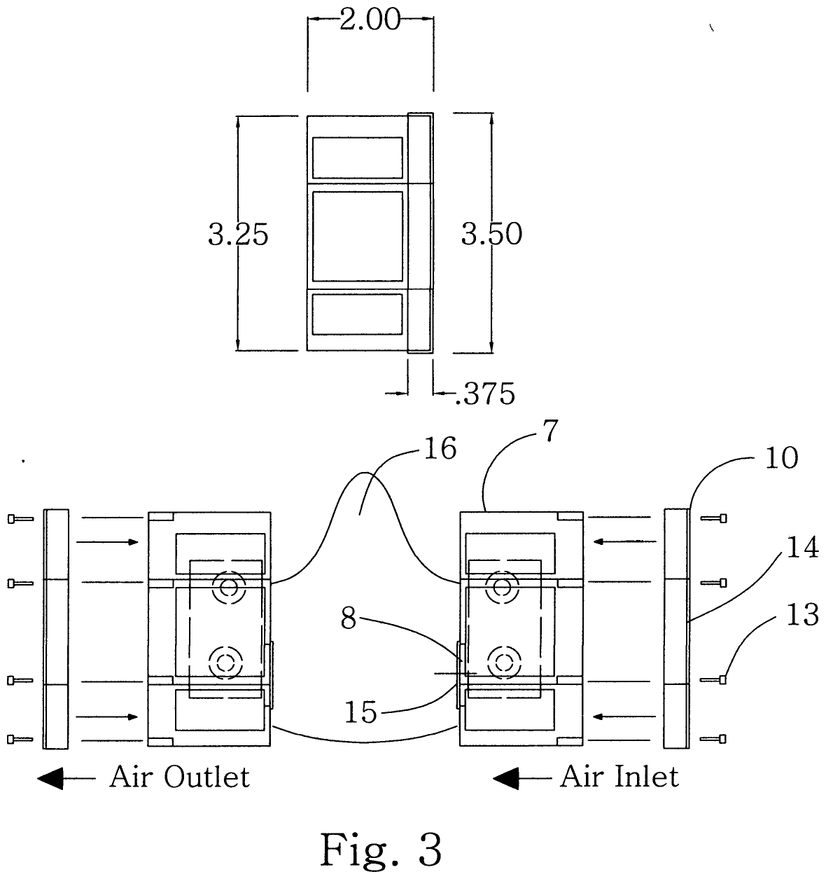

FIG. 2 illustrates a side view of the Ultra-V Mask light chamber depicting my design which is comprised of a chamber housing (7) which incorporates a filter (1) of which initial air flow occurs through from inlets (9) and a secondary filter (18) that will prevent any contaminants from entering the lungs incase of a element malfunction. This air flow is slowed down and disrupted by a series of baffles (2) in order to increase maximum volumetric sterilization of air. Unidirectional air flow is achieved by two check valves (8) that independently open and close as the user inhales and exhales. These check valves are installed in the inlet and outlet ports (15) of each chamber (7) as illustrated in FIG. 3. The air that passes through these chambers flows past two UVC light elements in each chamber (3) which are powered by a rechargeable 9V battery (6) that is controlled by a power switch (5) each chamber. System recharge is accomplished by the charge jacks (4) one per chamber.

FIG. 3 illustrates a front view of the light chambers of the Ultra-V Mask showing the chamber cover (10) which is attached to the chamber housing via four number eight screws (13) each chamber. Mating surfaces between (7) and (10) are sealed with a gasket (14). Light chamber (7) is attached to the face shroud (16) by a twist lock port (15). Unidirectional airflow is achieved by means of two directional check valves (8) installed one in each port (15).

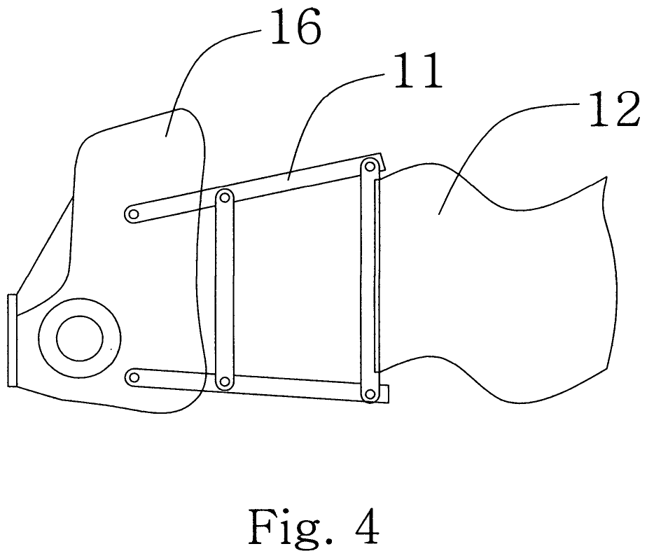

FIG. 4 depicts the premanufactured face shroud (16) Which is attached to the face by means of a mask harness (11) that is attached to a velcro headstrap (12). which comfortably secures around the back of the head instead of the ears.

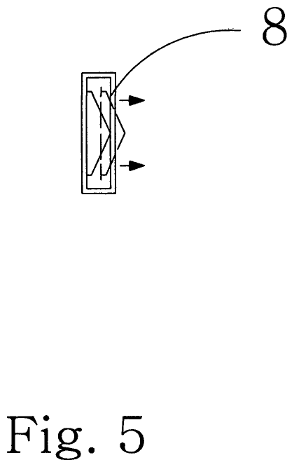

FIG. 5 illustrates the premanufactured check valve (8) which opens and closes as the user inhales and exhales.

1. This device treats directional air flow obtained by check valves (8) as illustrated in FIG. 3 with UVC light as it is inhaled and exhaled through the light chambers (7) containing two UVC elements (3) in each chamber.

2. Air flow is slowed and disrupted by a series of baffles (2). These baffles create turbulence which provides the UVC light with more time to destroy any airborne bacteria that may exist resulting in sterile air entering the lungs and sterile air exiting the lungs.

3. Power is supplied to the light elements (3) via two 9V rechargeable batteries (6) one in each light chamber (7) which are controlled by two switches (5).
